(19) United States
(12) Patent Application Publication
(10) Pub. No.: US 2017/0326100 A1
(43) Pub. Date: Nov. 16, 2017

(54) COMPOSITION FOR ACTIVATING LONGEVITY GENE

(71) Applicant: AMOREPACIFIC CORPORATION, Seoul (KR)

(72) Inventors: Hyun Seo KANG, Yongin-si, Gyeonggi-do (KR); Hyoung June KIM, Yongin-si, Gyeonggi-do (KR); Se Jin YOO, Yongin-si, Gyeonggi-do (KR); Ji Hyun KIM, Yongin-si, Gyeonggi-do (KR)

(21) Appl. No.: 15/534,625

(22) PCT Filed: Nov. 26, 2015
(86) PCT No.: PCT/KR2015/012773

§ 371 Date: Jun. 9, 2017

(30) Foreign Application Priority Data
Dec. 9, 2014 (KR) 10-2014-0175698
Dec. 9, 2014 (KR) 10-2014-0175699
Dec. 9, 2014 (KR) 10-2014-0175700
Dec. 9, 2014 (KR) 10-2014-0175701
Dec. 9, 2014 (KR) 10-2014-0175702

(51) Int. Cl. A61K 31/353; A61Q 19/08; A61K 8/49

(52) U.S. Cl. CPC A61K 31/353; A61K 8/49; A61Q 19/08

(57) ABSTRACT

Disclosed is a composition for activating one or more genes of an XPD gene, a Klotho gene, a Sirt-1 gene, an ERCC8 gene and a FoxO3 gene, which contains a methylated catechin, a salt thereof, a prodrug thereof, a hydrate thereof, a solvate thereof or an isomer thereof as an active ingredient. In an aspect, the present disclosure provides a pharmaceutical composition, a cosmetic composition or a food composition which activates one or more genes of an XPD gene, a Klotho gene, a Sirt-1 gene, an ERCC8 gene and a FoxO3 gene and is useful in preventing or treating disease related with the genes, preventing aging and improving skin.

### TECHNICAL FIELD

The present disclosure relates to a composition which contains a methylated catechin, a salt thereof, a prodrug thereof, a hydrate thereof, a solvate thereof or an isomer thereof as an active ingredient.

### BACKGROUND ART

Skin aging is an inevitable process for humans. However, not much is known about how the skin aging proceeds. In particular, researches on aging at individual levels are difficult because it takes a very long time.

Studies on skin aging have focused mainly on photoaging and intrinsic aging. With regard to photoaging, methods for blocking UV, which is the main cause, and preventing skin change caused by UV radiation have been studied actively. Also, methods for alleviating age-related intrinsic aging have been studied. Recently, focus is made on finding methods for regulating skin aging. In particular, methods for preventing skin aging based on researches on the genes that regulate the aging and life span of individuals are being studied.

### REFERENCES OF RELATED ART

Korean Patent Registration No. 10-0531947.

### DISCLOSURE

### Technical Problem

In an aspect, the present disclosure is directed to providing a composition for activating one or more genes of an XPD gene, a Klotho gene, a Sirt-1 gene, an ERCC8 gene and a FoxO3 gene as aging-related longevity genes using a methylated catechin.

In another aspect, the present disclosure is directed to providing a pharmaceutical composition, a cosmetic composition or a food composition for preventing or treating diseases related with an XPD gene, a Klotho gene, a Sirt-1 gene, an ERCC8 gene or a FoxO3 gene by activating one or more of the genes.

In another aspect, the present disclosure is directed to providing a pharmaceutical composition, a cosmetic composition or a food composition with superior antiaging and skin improving effects as well as superior safety for skin by activating one or more genes of an XPD gene, a Klotho gene, a Sirt-1 gene, an ERCC8 gene and a FoxO3 gene.

### Technical Solution

In an aspect, the present disclosure provides a composition for activating longevity genes, which contains a methylated catechin, a salt thereof, a prodrug thereof, a hydrate thereof, a solvate thereof or an isomer thereof as an active ingredient, wherein the longevity gene is one or more of an XPD gene, a Klotho gene, a Sirt-1 gene, an ERCC8 gene and a FoxO3 gene.

In an exemplary embodiment, the activation of the longevity gene may enhance transcription to mRNA.

In an exemplary embodiment, the methylated catechin may be extracted from green tea leaf.

In an exemplary embodiment, the methylated catechin may be represented by Chemical Formula 1:

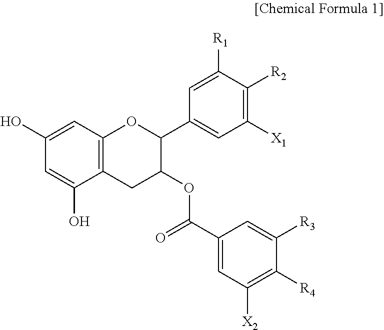

wherein each of R₁, R₂, R₃ and R₄ is independently OCH₃ or OH, except for the case where all of R₁, R₂, R₃ and R₄ are OH, and each of X₁ and X₂ is independently H or OH.

In an exemplary embodiment, the methylated catechin may be one or more selected from a group consisting of EGCG3″Me (epigallocatechin-3-O-(3-O-methyl)gallate), EGCG4″Me (epigallocatechin-3-O-(4-O-methyl)gallate), ECG3″Me (epicatechin-3-O-(3-O-methyl)gallate), ECG4″Me (epicatechin-3-O-(4-O-methyl)gallate), GCG3″Me (gallocatechin-3-O-(3-O-methyl)gallate), GCG4″Me (gallocatechin-3-O-(4-O-methyl)gallate), CG3″Me (catechin-3-O-(3-O-methyl)gallate) and CG4″Me (catechin-3-O-(4-O-methyl)gallate).

In an exemplary embodiment, the composition may contain 0.0001-10 wt % of a methylated catechin, a salt thereof, a prodrug thereof, a hydrate thereof, a solvate thereof or an isomer thereof based on the total weight of the composition.

In an exemplary embodiment, the composition may be for enhancing the expression of one or more protein of an XPD protein, a Klotho protein, a Sirt-1 protein, an ERCC8 protein and a FoxO3 protein.

In an exemplary embodiment, the composition may be for extending life span, delaying biological or skin aging or improving symptoms of biological or skin aging.

In an exemplary embodiment, the composition may be for enhancing skin elasticity or improving skin wrinkles.

In an exemplary embodiment, the composition may be for improving skin.

In an exemplary embodiment, the composition may be for moisturizing skin or strengthening skin barrier.

In an exemplary embodiment, the composition may be for preventing or treating a one or more disease of an XPD-related disease, a Klotho-related disease, a Sirt-1-related disease, an ERCC8-related disease and a FoxO3-related disease.

In an exemplary embodiment, the XPD-related disease may be cancer, xeroderma pigmentosum, Cockayne syndrome or trichothiodystrophy, the Klotho-related disease may be arteriosclerosis, osteoporosis, stroke or Alzheimer's disease, the Sirt-1-related disease may be cancer, diabetes, neurodegenerative disease, obesity, inflammatory disease or allergic respiratory disease, the ERCC8-related disease may be cancer or Cockayne syndrome, and the FoxO3-related disease may be cancer or inflammatory disease.

In an exemplary embodiment, the composition may be a pharmaceutical composition.

In an exemplary embodiment, the composition may be a cosmetic composition.

In an exemplary embodiment, the composition may be a food composition.

### Advantageous Effects

In an aspect, the present disclosure provides a composition which activates one or more genes of an XPD gene, a Klotho gene, a Sirt-1 gene, an ERCC8 gene and a FoxO3 gene, which are aging-related longevity genes, using a methylated catechin.

In another aspect, the present disclosure provides a pharmaceutical composition, a cosmetic composition or a food composition for preventing or treating diseases related with an XPD gene, a Klotho gene, a Sirt-1 gene, an ERCC8 gene or a FoxO3 gene by activating one or more of the genes.

In another aspect, the present disclosure provides a pharmaceutical composition, a cosmetic composition or a food composition with superior antiaging and skin improving effects as well as superior safety for skin by activating one or more genes of an XPD gene, a Klotho gene, a Sirt-1 gene, an ERCC8 gene and a FoxO3 gene.

### BRIEF DESCRIPTION OF DRAWINGS

FIG. 1 shows a result of comparing the effect of EGCG and EGCG″3Me on the differentiation of keratinocytes.

FIG. 2 shows the change in cell survival ratio of keratinocytes depending on the concentration of EGCG and EGCG3″Me.

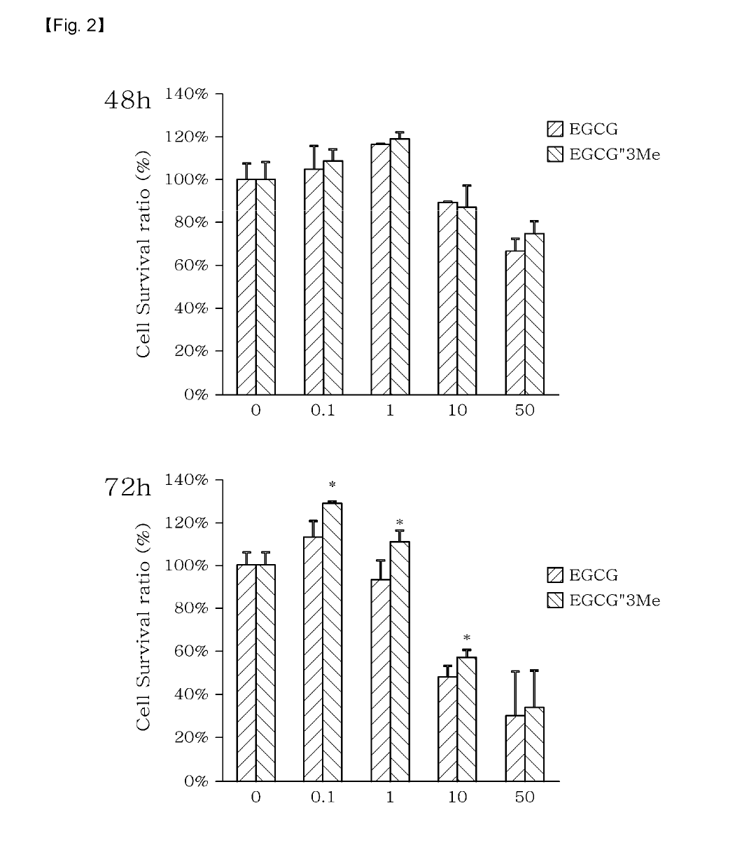

### BEST MODE

Hereinafter, the present disclosure is described in detail.

In an aspect, the present disclosure provides a composition for activating longevity genes, which contains a methylated catechin, a salt thereof, a prodrug thereof, a hydrate thereof, a solvate thereof or an isomer thereof as an active ingredient, wherein the longevity gene is one or more of an XPD gene, a Klotho gene, a Sirt-1 gene, an ERCC8 gene and a FoxO3 gene.

In an aspect, the present disclosure provides a method for activating one or more longevity genes of an XPD gene, a Klotho gene, a Sirt-1 gene, an ERCC8 gene and a FoxO3 gene, which includes a step of administering an effective amount of a methylated catechin, a salt thereof, a prodrug thereof, a hydrate thereof, a solvate thereof or an isomer thereof to a subject in need thereof.

In an aspect, the present disclosure provides a use of a methylated catechin, a salt thereof, a prodrug thereof, a hydrate thereof, a solvate thereof or an isomer thereof for preparing a composition for activating one or more longevity genes of an XPD gene, a Klotho gene, a Sirt-1 gene, an ERCC8 gene and a FoxO3 gene.

In an aspect, the present disclosure provides a methylated catechin, a salt thereof, a prodrug thereof, a hydrate thereof, a solvate thereof or an isomer thereof for activating one or more longevity genes of an XPD gene, a Klotho gene, a Sirt-1 gene, an ERCC8 gene and a FoxO3 gene.

In the present disclosure, a “salt” or a “pharmaceutically acceptable salt” refers to a salt according to the present disclosure which is pharmaceutically acceptable and has a desired pharmacological activity of a parent compound. It includes a common salt formed from an inorganic acid, an organic acid, an inorganic base or an organic base and a quaternary ammonium acid addition salt. The salt may include (1) an acid addition salt formed from an inorganic acid such as hydrochloric acid, hydrobromic acid, sulfuric acid, nitric acid, phosphoric acid, etc. or from an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tert-butylacetic acid, lauryl sulfuric acid, gluconic acid, glutamic acid, hydroxynaphthoic acid, salicylic acid, stearic acid and muconic acid or (2) a salt formed when an acidic proton present in the parent compound is substituted. Specific examples of a suitable base salt include salts of sodium, lithium, potassium, magnesium, aluminum, calcium, zinc, N,N′-dibenzylethylenediamine, chloroprocaine, choline, diethanolamine, ethylenediamine, N-methylglucosamine and procaine.

In the present disclosure, “pharmaceutically acceptable” means approved by a regulatory agency of a government or an international organization corresponding thereto or listed in the Pharmacopoeia or other generally recognized pharmacopoeia for use in animals, more specifically in humans, since significant toxic effect can be avoided when used with a common medicinal dosage.

In the present disclosure, a “prodrug” refers to a drug whose physical and chemical properties have been changed such that it does not exhibit physiological activity as it is but exerts medicinal effect after it is converted to the original drug through chemical or enzymatic action in vivo. After being administered, the prodrug is chemically converted to an active drug through metabolism. In general, the prodrug is a functional derivative of the compound of the present disclosure and is easily converted to the desired compound in vivo. Methods for selecting and preparing a suitable prodrug derivative are described, for example, in “Design of Prodrugs”, H Bund Saard, Elsevier, 1985, the entire contents of which are incorporated herein by reference.

In the present disclosure, a “hydrate” refers to a compound to which water is bound. The term is used in a broad sense, including an inclusion compound which lacks chemical binding between water and the compound.

In the present disclosure, a “solvate” refers to a higher-order compound formed between a solute molecule or ion and a solvent molecule or ion.

In the present disclosure, an “isomer” refers to a compound which has the same chemical formula but is not identical. Isomers include structural isomers, geometric isomers, optical isomers and stereoisomers. The structural isomers refer to the compounds which have the same molecular but have different properties because of different structures. The geometric isomers refer to the isomers which have different spatial arrangement of atoms or a group of atoms bound to two atoms connected by a double bond. The stereoisomers refer to the compounds which have the same chemical structure but are different in the spatial arrangement of atoms or substituents. The optical isomers (enantiomers) refer to two stereoisomers which are non-superimposable mirror images of each other. The diastereomers refer to the stereoisomers that have two or more chiral centers and are not mirror images of each other.

In the present disclosure, the “isomers” include, in particular, not only the optical isomers (e.g., essentially pure enantiomers, essentially pure diastereomers or mixtures thereof) but also conformational isomers (the isomers that are different only in the angle of one or more chemical bond), positional isomers (particularly, tautomers) or geometric isomers (e.g., cis-trans isomers).

In the present disclosure, “essentially pure” means, for example, when used in connection with enantiomers or diastereomers, that a specific compound such as the enantiomer or the diastereomer, is present in about 90% (w/w) or more, specifically about 95% or more, more specifically about 97% or more or about 98% or more, further more specifically about 99% or more, even more specifically about 99.5% or more.

In the present disclosure, “activating a gene” means promotion of transcription of a specific gene on chromosomal DNA and translation into a protein so that its function can be exerted. That is to say, it means promotion of the expression of the gene so that the transcription to mRNA and the translation to the protein occur actively and the function of the gene can be exerted well.

The XPD (ERCC2; excision repair cross-complementation group 2) protein is a member of DNA repair proteins that maintain the integrity of DNA. It is one of two enzymes involved in DNA unfolding and performs nucleotide excision repair with the other XP protein. Therefore, damage to the XPD gene can cause various skin diseases and aging (Mol Cell. 2003 June; 11(6): 1635-46.). In human, the XPD gene is located on 45.85-45.87 Mb of chromosome 19 and has an mRNA sequence of, for example, NM_000400. And, the peptide sequence is NP_000391. Defect in DNA repair causes aging-related diseases (Best, BP (2009). “Nuclear DNA damage as a direct cause of aging”. Rejuvenation Research 12(3): 199-208.) and increases the risk of cancer (Bernstein C, Bernstein H, Payne C M, Garewal H. DNA repair/pro-apoptotic dual-role proteins in five major DNA repair pathways: fail-safe protection against carcinogenesis. Mutat Res. 2002 June; 511(2): 145-78. Review.) by accelerating aging. Mutation of the XPD gene which is a DNA repair protein affecting the defect in DNA repair may cause xeroderma pigmentosum, Cockayne syndrome or trichothiodystrophy. Xeroderma pigmentosum is a recessive hyperphotosensitive skin disease with high incidence of skin cancer and is caused by the mutation of DNA repair-related genes. Cockayne syndrome is a type of dwarfism characterized by growth failure, hyperphotosensitivity or premature aging. This disease is also known to be caused by the defect in DNA repair genes. Because the genes causing Cockayne syndrome are also involved in protein production, abnormal accumulation and production of proteins may occur. There are four forms of Cockayne syndrome, some of which show symptoms associated with xeroderma pigmentosum. Trichothiodystrophy is a sulfur-defective hair dystrophy characterized by brittle and easily breaking hair due to insufficient production of sulfur-containing proteins. The XPD protein, which is one of the DNA repair proteins, is known as the common cause of these three diseases are. In an exemplary embodiment, the methylated catechin may be extracted from green tea leaf. It may be extracted with cold water or warm water after washing green tea leaf. Specifically, an extract obtained using warm water may be used after solidifying into powder.

Klotho is an enzyme encoded by the KL gene. This gene encodes a type-I membrane protein that is related to β-glucuronidases. In human, the klotho gene is located on 33.59-33.64 Mb of chromosome 13 and has an mRNA sequence of, for example, NM_004795. And, the peptide sequence is NP_004786.

Klotho knock-out mice manifest various symptoms resembling accelerated aging and exhibit arteriosclerosis related with increased level of 1,25(OH)₂D₃, vascular calcification, soft tissue calcification, emphysema, hypoactivity, gonadal dysgenesis, infertility, skin atrophy, ataxia, hypoglycemia and hyperphosphatemia (Mutation of the mouse klotho gene leads to a syndrome resembling ageing. Nature 1997; 390, 45-51). On the contrary, increased expression of the klotho protein leads to longer life span, increased insulin resistance, increased IGF-1 resistance, etc. (Kurosu et al., 2005).

It has been reported that the single nucleotide polymorphism of the longevity gene Klotho is associated with shortened life span, osteoporosis, stroke and coronary artery disease in human, too (Arking et al., 2002, Kawano et al., 2002; Mullin et al., 2005, Ogata et al., 2002; Yamada et al., 2005). In addition, it is reported that higher level of the Klotho protein leads to extended life span of brain cells, decreased incidence of related diseases such as cardiac diseases and strengthened cognitive ability such as attention, memory, perception, etc. and that shortage of the protein accelerates the aging process. However, the relationship between skin cells and Klotho expression or a substance that can increase Klotho expression has not been studied yet.

SIRT1 (silent mating type information regulation 2 homolog; sirtuin 1) is an NAD⁺-dependent deacetylase. In human, the Sirt-1 gene is located on 69.64-69.68 Mb of chromosome 10 and has an mRNA sequence of, for example, NM_001142498. And, the peptide sequence is NP_001135970. It is known as an enzyme which regulates the function of various proteins by deacetylating the lysine residue (Ageing Res, Vol. 1, pp. 313-326, (2002)) and is known to exhibit an effect of inhibiting death of aged ells.

A research team at Harvard Medical School reported that the reason why reduction in diet leads to extended life span is because the activity of Sirt-1 is increased (Science. 2004 Jul. 16; 305(5682): 390-2. Epub 2004 Jun. 17.). It is very similar to yeast Sir2, which has NAD⁺-dependent class III histone deacetylation activity. In particular, it regulates the function of transcription factors such as nuclear factor-kB, p53, etc. by removing the acetyl group (Cancer Res, Vol. 64, pp. 7513-7525, (2004); Cell, Vol. 107, pp. 149-159, (2001); Trends Endocrinol Metab, Vol. 17, pp. 186-191, (2006)).

SIRT1 is involved in reconstitution of chromatin related with gene expression, DNA damage, extension of life span related with reduced diet, etc. (Chen et al., Science 310, 1641, 2005). Also, SIRT1 is known to be related with allergic respiratory diseases (J Allergy Clin Immunol. 2010 February; 125(2): 449-460. e14. doi: 10.1016/j.jaci.2009.08.009. Epub 2009 Oct. 27.). Like yeast Sir2, SIRT1 reconstitutes chromatin and inhibits gene expression through histone deacetylation. In addition to the histone protein, it induces deacetylation of various transcription factors involved in cellular growth, stress response, endocrine regulation, etc.

A method of applying the increase in deacetylation activity by SIRT1 for diabetes, obesity, neurodegenerative diseases, aging-related diseases, etc. has been reported recently. That is to say, it has been reported that SIRT1 regulates the growth, aging and death of cells by being involved in gene expression, sugar metabolism, insulin production, inflammatory response, protection of brain cells, etc. and, in tissue and individual levels, is involved in various aging-related diseases such as cancers, metabolic diseases, obesity, inflammatory diseases, diabetes, cardiac diseases, neurodegenerative diseases, etc.

ERCC8 (excision repair cross-complementation group 8) is a protein which plays an important role in DNA repair. In human, the ERCC8 gene is located on 60.17-60.24 Mb of chromosome 5 and has an mRNA sequence of, for example, NM_000082. And, the peptide sequence is NP_000073. Mutations in ERCC8 can lead to Cockayne syndrome which is a genetic disease accompanied by premature aging. The premature aging reveals that ERCC8 significantly affects aging.

Defect in DNA repair causes aging-related diseases by accelerating aging (Best, BP (2009). “Nuclear DNA damage as a direct cause of aging”. Rejuvenation Research 12(3): 199-208.) and increases the incidence of cancer (Bernstein C, Bernstein H, Payne C M, Garewal H. DNA repair/pro-apoptotic dual-role proteins in five major DNA repair pathways: fail-safe protection against carcinogenesis. Mutat Res. 2002 June; 511(2): 145-78. Review.).

FoxO3a is a protein encoded by the FoxO3 (forkhead box 03) gene which is known as a longevity gene. It is a transcription factor involved in insulin signaling and acts on the expression of enzymes such as Mn-SOD and catalase. In human, the FoxO3 gene is located on 108.88-109.01 Mb of chromosome 6 and has an mRNA sequence of, for example, NM_001455. And, the peptide sequence is NP_001446. Activation of FoxO3a leads to antiaging effect through, for example, the activation of a defensive mechanism in vivo.

The FoxO3 protein is known as an anticancer agent (Myatt S S, Lam E W (November 2007). “The emerging roles of forkhead box (Fox) proteins in cancer”. Nat. Rev. Cancer 7 (11): 847-59.). The activity of the FoxO3 gene is related with carcinogenesis. Downregulation of FoxO3 activity is often seen in cancer and the FoxO3 gene is also known to be relevant to inflammatory disease through proliferation of lymphocytes (Immunity 2004. 21: 203-213., Proc. Natl. Acad. Sci. 2004. 101: 2975-2980., Cell 1999. 96: 857-868).

In an exemplary embodiment, the methylated catechin may be extracted from green tea leaf. It may be extracted with cold water or warm water after washing green tea leaf. Specifically, an extract obtained using warm water may be used after solidifying into powder.

In an exemplary embodiment, the methylated catechin may be one or more selected from a group consisting of methylated epigallocatechin gallate (EGCG), methylated gallocatechin gallate (GCG), methylated epigallocatechin (EGC), methylated epicatechin gallate (ECG), methylated gallocatechin (GC), methylated catechin gallate (CG), methylated epicatechin (EC) and methylated catechin (C).

In an exemplary embodiment, the methylated catechin may be represented by Chemical Formula 1:

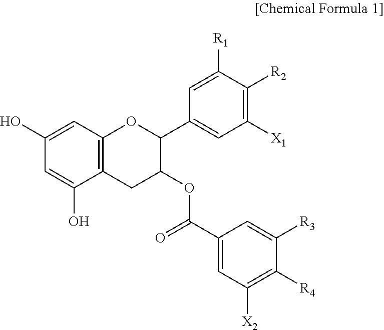

wherein each of R₁, R₂, R₃ and R₄ is independently OCH₃ or OH, except for the case where all of R₁, R₂, R₃ and R₄ are OH, and each of X₁ and X₂ is independently H or OH.

In an exemplary embodiment, the methylated catechin may be one or more selected from a group consisting of EGCG3″Me (epigallocatechin-3-O-(3-O-methyl)gallate), EGCG4″Me (epigallocatechin-3-O-(4-O-methyl)gallate), ECG3″Me (epicatechin-3-O-(3-O-methyl)gallate), ECG4″Me (epicatechin-3-O-(4-O-methyl)gallate), GCG3″Me (gallocatechin-3-O-(3-O-methyl)gallate), GCG4″Me (gallocatechin-3-O-(4-O-methyl)gallate), CG3″Me (catechin-3-O-(3-O-methyl)gallate) and CG4″Me (catechin-3-O-(4-O-methyl)gallate).

In an exemplary embodiment, the composition may contain 0.0001-10 wt % or 0.001-1 wt % of a methylated catechin, a salt thereof, a prodrug thereof, a hydrate thereof, a solvate thereof or an isomer thereof based on the total weight of the composition.

In an exemplary embodiment, the composition may be for enhancing the expression of one or more protein of an XPD protein, a Klotho protein, a Sirt-1 protein, an ERCC8 protein and a FoxO3 protein. When skin cells are treated with the methylated catechin, superior antiaging and skin improving effects are exhibited as the expression of one or more protein of an XPD protein, a Klotho protein, a Sirt-1 protein, an ERCC8 protein and a FoxO3 protein is enhanced.

In an exemplary embodiment, the composition may be for extending life span, delaying biological or skin aging or improving symptoms of biological or skin aging.

In an exemplary embodiment, the composition may be for enhancing skin elasticity or improving skin wrinkles.

In an exemplary embodiment, the composition may be for improving skin.

In an exemplary embodiment, the composition may be for fighting against cancer.

In an exemplary embodiment, the composition may be for preventing or treating an XPD-related disease. The XPD-related disease refers to a disease caused by XPD which is a DNA repair protein affecting defect in DNA repair and includes xeroderma pigmentosum, Cockayne syndrome or trichothiodystrophy. In an exemplary embodiment, the composition may be a pharmaceutical composition. The pharmaceutical composition may be for antiaging, for improving skin or for preventing or treating cancer, xeroderma pigmentosum, Cockayne syndrome or trichothiodystrophy.

In an exemplary embodiment, the composition may be for preventing or treating a klotho-related disease. The klotho-related disease refers to a disease caused by klotho, e.g., deficiency of the klotho protein. Specific examples include arteriosclerosis, osteoporosis, stroke, Alzheimer's disease, etc. In an exemplary embodiment, the composition may be for preventing or treating arteriosclerosis, osteoporosis, stroke or Alzheimer's disease. In an exemplary embodiment, the composition may be a pharmaceutical composition. The pharmaceutical composition may be for antiaging, for improving skin or for preventing or treating arteriosclerosis, osteoporosis, stroke or Alzheimer's disease.

In an exemplary embodiment, the composition may be for preventing or treating a Sirt-1-related disease. The Sirt-1-related disease refers to a disease caused by Sirt-1, e.g., deficiency, inhibition, etc. of the Sirt-1 protein which is an enzyme that regulates the function of various proteins by deacetylating the lysine residue and includes cancer, diabetes, neurodegenerative disease, obesity, inflammatory disease, allergic respiratory disease, etc. In an exemplary embodiment, the composition may be for preventing or treating cancer. In an exemplary embodiment, the composition may be for preventing or treating diabetes. In an exemplary embodiment, the composition may be for preventing or treating neurodegenerative diseases. Examples of the neurodegenerative disease include Alzheimer's disease, amyotrophic lateral sclerosis, Parkinson's disease, Huntington's disease, multiple sclerosis, etc. In an exemplary embodiment, the composition may be for preventing or treating obesity. In an exemplary embodiment, the composition may be for preventing or treating inflammatory diseases. Examples of the inflammatory disease include dermatitis, allergy, conjunctivitis, gingivitis, rhinitis, otitis media, pharyngitis, tonsillitis, pneumonia, gastric ulcer, duodenal ulcer, hepatitis, esophagitis, gastritis, enteritis, pancreatitis, colitis, nephritis, arthritis, generalized edema, localized edema, etc. In an exemplary embodiment, the composition may be a pharmaceutical composition. The pharmaceutical composition may be for antiaging, for improving skin or for preventing or treating cancer, diabetes, neurodegenerative diseases, obesity, inflammatory diseases or allergic respiratory diseases.

In an exemplary embodiment, the composition may be for preventing or treating an ERCC8-related disease. The ERCC8-related disease refers to a disease caused by ERCC8 which is a DNA repair protein affecting defect in DNA repair. Specific examples include aging-related diseases, cancer, Cockayne syndrome, etc. In an exemplary embodiment, the composition may be a pharmaceutical composition. The pharmaceutical composition may be for antiaging, for improving skin or for preventing or treating cancer or Cockayne syndrome.

In an exemplary embodiment, the composition may be for preventing or treating a FoxO3-related disease. The FoxO3-related disease refers to a disease caused by the activation or inhibition of the FoxO3 gene and includes cancer, aging-related diseases, inflammatory diseases, etc. Examples of the inflammatory disease include dermatitis, allergy, conjunctivitis, gingivitis, rhinitis, otitis media, pharyngitis, tonsillitis, pneumonia, gastric ulcer, duodenal ulcer, hepatitis, esophagitis, gastritis, enteritis, pancreatitis, colitis, nephritis, arthritis, generalized edema, localized edema, etc. In an exemplary embodiment, the composition may be a pharmaceutical composition. The pharmaceutical composition may be for antiaging, for improving skin or for preventing or treating cancer or inflammatory diseases.

In an aspect, the pharmaceutical composition may further contain a suitable carrier, excipient or diluent commonly used in the preparation of a pharmaceutical composition. In an aspect, examples of the carrier, excipient or diluent that can be contained in the composition include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, etc.

The pharmaceutical composition may be prepared into a formulation for oral administration such as a powder, a granule, a tablet, a capsule, a suspension, an emulsion, a syrup, an aerosol, etc., a formulation for external application, a suppository or a sterile injectable solution according to a commonly employed method.

The formulation may contain a commonly used diluent, excipient, etc. such as a filler, an extender, a binder, a wetting agent, a disintegrant, a surfactant, etc. A solid formulation for oral administration may include a tablet, a pill, a powder, a granule, a capsule, etc. The solid formulation may further include, in addition to the active ingredient, at least one excipient, e.g., starch, calcium carbonate, sucrose, lactose or gelatin. In addition to the simple excipient, a lubricant such as magnesium stearate or talc may also be contained. A liquid formulation for oral administration includes a suspension, a solution for internal use, an emulsion, a syrup, etc. and may contain, in addition to a commonly used simple diluent such as water and liquid paraffin, various excipients, e.g., a wetting agent, a sweetener, an aromatic, a preservative, etc. A formulation for parenteral administration may include a sterilized aqueous solution, a nonaqueous solution, a suspension, an emulsion, a freeze-dried formulation and a suppository. For the nonaqueous solution or the suspension, propylene glycol, polyethylene glycol, a vegetable oil such as olive oil, an injectable ester such as ethyl oleate, etc. may be used. As a base of the suppository, witepsol, macrogol, tween 61, laurin butter, cocoa butter, glycerogelatin, etc. may be used.

The administration dosage of the active ingredient disclosed in the present disclosure may vary depending on the physical condition and body weight of a patient, severity of a disease, drug type, and period and route of administration. The administration dosage may be selected in a range commonly used in the art. In an aspect, a daily administration dosage of the active ingredient may be 0.0001-1000 g/kg based on dry weight. In another aspect, the administration dosage may be 0.001-100 g/kg. In another aspect, the administration dosage may be 0.001-10 g/kg. In another aspect, the administration dosage may be 0.001-1 g/kg. In an aspect, the daily administration dosage may be 0.0001 g/kg or more, 0.001 g/kg or more, 0.05 g/kg or more, 0.01 g/kg or more or 0.05 g/kg or more. In another aspect, the daily administration dosage may be 500 g/kg or less, 100 g/kg or less, 50 g/kg or less, 10 g/kg or less, 1 g/kg or less or 0.5 g/kg or less. In an exemplary embodiment, the active ingredient may be administered at a daily dosage of about 0.086 g/kg. In another exemplary embodiment, it may be administered at a daily dosage of about 0.143 g/kg. The administration may be made once in 1-5 days or several times a day. In an aspect, the administration may be made 3 times a day.

The active ingredient disclosed in the present disclosure may be administered to mammals such as cattle, human, etc. through various routes. Any mode of administration may be expected. For example, it may be administered orally, rectally, intravenously, intramuscularly, subcutaneously, intrauterinely or intracerebroventricularly.

In an exemplary embodiment, the composition may be a cosmetic composition. The cosmetic composition may contain, in addition to the methylated catechin or the isomer thereof as the active ingredient, ingredients commonly used in a cosmetic composition. For example, it may contain a common adjuvant such as an antioxidant, a stabilizer, a solubilizer, a vitamin, a pigment, a colorant and a fragrance as well as a carrier.

The cosmetic composition of the present disclosure may be prepared into any formulation common in the art. For example, it may be prepared into a solution, a suspension, an emulsion, a paste, a gel, a cream, a lotion, a powder, a soap, a surfactant-containing cleanser, an oil, a powder foundation, an emulsion foundation, a wax foundation, a spray, etc., although not being limited thereto. More specifically, it may be prepared into a makeup cosmetic such as a softening lotion, a nourishing lotion, a lotion, a body lotion, a nourishing cream, a massage cream, a moisturizing cream, a hand cream, an essence, an eye cream, a cleansing cream, a cleansing foam, a cleansing water, a pack, a gel, a patch, a spray, a powder, an oil-in-water (0/W) or water-in-oil (W/O) base cosmetic, a lipstick, a makeup base, a foundation, etc. or a cleanser such as a shampoo, a rinse, a body cleanser, a toothpaste, a mouthwash, etc., a hair fixative such as a hair conditioner, a gel, a mousse, etc. or a hair cosmetic such as a tonic, a hair dye, etc.

When the formulation of the cosmetic composition of the present disclosure is a paste, a cream or a gel, an animal oil, a plant oil, a wax, a paraffin, a starch, tragacanth, a cellulose derivative, polyethylene glycol, silicone, bentonite, silica, talc, zinc oxide, etc. may be used as a carrier.

When the formulation of the cosmetic composition of the present disclosure is a powder or a spray, lactose, talc, silica, aluminum hydroxide, calcium silicate or polyamide powder may be used as a carrier. Especially, the spray may further contain a propellant such as a chlorofluorohydrocarbon, propane/butane or dimethyl ether.

When the formulation of the cosmetic composition of the present disclosure is a solution or an emulsion, a solvent, a solubilizer or an emulsifier may be used as a carrier. Examples include water, ethanol, isopropanol, ethyl carbonate, ethyl acetate, benzyl alcohol, benzyl benzoate, propylene glycol, butylene glycol, 1,3-butyl glycol oil, polyoxyethylene hydrogenated castor oil, glycerol, glycerin, an aliphatic ester, phenoxyethanol, triethanolamine, polyethylene glycol, beeswax, polysorbate 60, sorbitan sesquioleate, paraffin, sorbitan stearate, lipophilic glyceryl monostearate, stearic acid, glyceryl stearate/PEG-400 stearate, a carboxyl polymer, sitosterol, polyglyceryl-2 oleate, a ceramide, cholesterol, steareth-4, dicetyl phosphate, macadamia oil, a carboxyvinyl polymer, xanthan gum, a fatty acid ester of sorbitan, etc.

When the formulation of the cosmetic composition of the present disclosure is a suspension, a liquid diluent such as water, ethanol, butylene glycol or propylene glycol, a suspending agent such as ethoxylated isostearyl alcohol, polyoxyethylene sorbitol ester and polyoxyethylene sorbitan ester, microcrystalline cellulose, hydroxyethyl cellulose, sodium hyaluronate, phenoxyethanol, aluminum metahydroxide, bentonite, agar, tragacanth, etc. may be used as a carrier.

When the formulation of the cosmetic composition of the present disclosure is a surfactant-containing cleanser, an aliphatic alcohol sulfate, an aliphatic alcohol ether sulfate, sulfosuccinic acid monoester, an isethionate, an imidazolinium derivative, methyl taurate, a sarcosinate, a fatty acid amide ether sulfate, an alkyl amidobetaine, an aliphatic alcohol, a fatty acid glyceride, a fatty acid diethanolamide, a vegetable oil, a lanolin derivative, an ethoxylated glycerol fatty acid ester, etc. may be used as a carrier.

In an exemplary embodiment, the composition may be a food composition. The food composition may be for antiaging, for improving skin or for preventing or ameliorating cancer, xeroderma pigmentosum, Cockayne syndrome, trichothiodystrophy, inflammatory disease, arteriosclerosis, osteoporosis, stroke, Alzheimer's disease, diabetes, neurodegenerative diseases, obesity or allergic respiratory diseases.

In an aspect, the food composition may be, for example, various foods, drinks, gum, tea, vitamin complexes, health supplement foods, etc. and may be used in the form of a powder, a granule, a tablet, a capsule or a drink. Each formulation of the food composition may contain, in addition to the active ingredient, ingredients commonly used in the art which can be easily selected by those skilled in the art without difficulty considering the particular formulation or use of purpose. These ingredients may provide a synergic effect.

The amount of the active ingredient contained in the food or drink composition may be, in general, 1-5 wt % for a health food composition and 0.02-10 g, specifically 0.3-1 g, per 100 mL for a health drink composition.

A liquid ingredient that may be contained in the health drink composition in addition to the active ingredient disclosed in the present disclosure is not particularly limited. Various flavors, natural carbohydrates, may be further contained as in common drinks. Examples of the natural carbohydrate include monosaccharides such as glucose, fructose, etc., disaccharides such as maltose, sucrose, etc., polysaccharides, sugars such as dextrin, cyclodextrin, etc., sugar alcohols such as xylitol, sorbitol, erythritol, etc., and so forth. As the flavor, a natural flavor (thaumatin, stevia extract (e.g., rebaudioside A, glycyrrhizin, etc.) or a synthetic flavor (e.g., saccharin, aspartame, etc.) may be used. The natural carbohydrate may be contained in an amount of generally about 1-20 g, specifically about 5-12 g, per 100 mL of the composition disclosed in the present disclosure.

Furthermore, in an aspect, the food composition may contain various nutrients, vitamins, minerals (electrolytes), flavors such as synthetic flavors and natural flavors, colorants, extenders (cheese, chocolate, etc.), pectic acid and its salts, alginic acid and its salts, organic acids, protective colloidal thickeners, pH control agents, stabilizers, antiseptics, glycerin, alcohols, carbonating agents used in carbonated drinks, and so forth. It may further contain pulp for preparing natural fruit juice or vegetable juice. These ingredients may be used independently or in combination. The addition amount of these additives is not particularly limited. In general, they are contained in an amount of about 0-20 parts by weight based on 100 parts by weight of the composition disclosed in the present disclosure.

### MODE FOR INVENTION

Hereinafter, the present disclosure will be described in detail through examples. However, the following examples are for illustrative purposes only and it will be apparent to those of ordinary skill in the art that the scope of the present disclosure is not limited by the examples.

### Test Example

There are three types of cells that constitute human skin. They are keratinocytes which make up the epidermis, melanocytes which produce melanin and fibroblasts which make up the dermis.

The keratinocytes are deeply involved in skin miniaturization by preventing evaporation of water and barrier function of protecting the skin from harmful factors. The melanocytes determine the color and tone of skin and also produces freckles and live spots. The fibroblasts produce elastic fibers such as collagen and are deeply associated with skin elasticity and skin wrinkles.

Experiment was conducted using normal human keratinocytes (NHK) and normal human fibroblasts (NHF) to investigate the effect of a methylated catechin. Specifically, 2×10⁵ NHFs (normal human fibroblasts) purchased from Lonza (Allendale, N.J., USA) were cultured on a 60-mm dish at 37° C. for 24 hours using DMEM. The medium was discarded and the cells were transferred to a new tissue culture flask. Also, NHEKs (normal human epidermal keratinocytes, corresponding to NHK) purchased from Lonza (Allendale, N.J., USA) were cultured using a keratinocyte growth medium (KGM-GOLD, Lonza, Allendale, N.J., USA). The cells were detached with 0.025% trypsin and transferred to a new tissue culture flask after subculturing.

As described below, it was confirmed that a composition containing a methylated catechin as an active ingredient leads to increased expression of longevity genes (XPD, Klotho, Sirt-1, ERCC8 and Fox03) in the keratinocytes and the fibroblasts. Also, the effect of the expression of the longevity genes (XPD, Klotho, Sirt-1, ERCC8 and Fox03) on the increased differentiation of keratinocytes and the increased extracellular matrix (ECM) in fibroblasts was investigated.

(1) Evaluation of XPD Activation

The effect of a methylated catechin on XPD activation was compared with those of retinol and an unmethylated catechin.

Specifically, after treating the keratinocytes (NHK) and the fibroblasts (NHF) with each of retinol, green tea EGCG and green tea EGCG″3Me at 10 ppm, followed by incubation at 37° C. for 24 hours, total RNA was isolated from the cells and the relative expression of XPD mRNA was compared.

The total RNA was isolated using TRIzol™ (Invitrogen, Carlsbad, Calif., USA) according to the manufacturer's protocol. RNA concentration was measured spectrophotometrically and RNA integrity was measured using BioAnalyzer 2100 (Agilent Technologies, Santa Clara, Calif., USA). 4 μg of RNA was reverse transcribed to cDNA using SuperScript®III reverse transcriptase (Invitrogen, Carlsbad, Calif., USA). The cDNA was stored at −70° C. The expression level of the target gene was measured by quantitative real-time TaqMan RT-PCR (7500Fast, Applied Biosystems, Foster City, Calif., USA). The cycle condition was 10 minutes at 95° C., 50 cycles of 15 minutes at 95° C. and 1 minute at 60° C.

TABLE 1
Experiment on keratinocytes: relative expression of XPD mRNA

| | |
|---|---|
| Control (none) | 1.0 |
| Retinol | 10.1 |
| Green tea EGCG (10 ppm) | 9.2 |
| Green tea EGCG3″Me (10 ppm) | 21.4 |

TABLE 2
Experiment on fibroblasts: relative expression of XPD mRNA

| | |
|---|---|
| Control (none) | 1.0 |
| Retinol | 2.4 |
| Green tea EGCG (10 ppm) | 2.8 |
| Green tea EGCG3″Me (10 ppm) | 6.9 |

It was confirmed that the composition containing the methylated catechin as an active ingredient remarkably increases the expression of the XPD gene as compared to the unmethylated catechin.

(2) Evaluation of Klotho Activation

The effect of a methylated catechin on Klotho activation was compared with those of retinol and an unmethylated catechin.

Specifically, after treating the keratinocytes (NHK) and the fibroblasts (NHF) with each of retinol (10 ppm), green tea EGCG (1 and 10 ppm) and green tea EGCG″3Me (1 and 10 ppm), followed by incubation at 37° C. for 24 hours, total RNA was isolated from the cells and the relative expression of Klotho mRNA was compared.

The total RNA was isolated using TRIzol™ (Invitrogen, Carlsbad, Calif., USA) according to the manufacturer's protocol. RNA concentration was measured spectrophotometrically and RNA integrity was measured using BioAnalyzer 2100 (Agilent Technologies, Santa Clara, Calif., USA). 4 μg of RNA was reverse transcribed to cDNA using SuperScript®III reverse transcriptase (Invitrogen, Carlsbad, Calif., USA). The cDNA was stored at −70° C. The expression level of the target gene was measured by quantitative real-time TaqMan RT-PCR (7500Fast, Applied Biosystems, Foster City, Calif., USA). The cycle condition was 10 minutes at 95° C., 50 cycles of 15 minutes at 95° C. and 1 minute at 60° C.

TABLE 3
Experiment on keratinocytes: relative expression of Klotho mRNA

| | |
|---|---|
| Control (none) | 1.0 |
| Retinol | 9.8 |
| Green tea EGCG (1 ppm) | 2.5 |
| Green tea EGCG (10 ppm) | 10.2 |
| Green tea EGCG3″Me (1 ppm) | 4.2 |
| Green tea EGCG3″Me (10 ppm) | 10.5 |

TABLE 4
Experiment on fibroblasts: relative expression of Klotho mRNA

| | |
|---|---|
| Control (none) | 1.0 |
| Retinol | 2.2 |
| Green tea EGCG (1 ppm) | 1.2 |
| Green tea EGCG (10 ppm) | 1.8 |
| Green tea EGCG3″Me (1 ppm) | 1.7 |
| Green tea EGCG3″Me (10 ppm) | 2.1 |

It was confirmed that the composition containing the methylated catechin as an active ingredient increases the expression of the Klotho gene as compared to the unmethylated catechin. In particular, the difference was distinct at low concentration. Accordingly, it can be seen that the methylated catechin is superior in terms of economy and efficiency.

(3) Evaluation of Sirt-1 Activation

The effect of a methylated catechin on Sirt-1 activation was compared with those of retinol and an unmethylated catechin.

Specifically, after treating the keratinocytes (NHK) and the fibroblasts (NHF) with each of retinol, green tea EGCG and green tea EGCG″3Me at 10 ppm, followed by incubation at 37° C. for 24 hours, total RNA was isolated from the cells and the relative expression of Sirt-1 mRNA was compared.

The total RNA was isolated using TRIzol™ (Invitrogen, Carlsbad, Calif., USA) according to the manufacturer's protocol. RNA concentration was measured spectrophotometrically and RNA integrity was measured using BioAnalyzer 2100 (Agilent Technologies, Santa Clara, Calif., USA). 4 μg of RNA was reverse transcribed to cDNA using SuperScript®III reverse transcriptase (Invitrogen, Carlsbad, Calif., USA). The cDNA was stored at −70° C. The expression level of the target gene was measured by quantitative real-time TaqMan RT-PCR (7500Fast, Applied Biosystems, Foster City, Calif., USA). The cycle condition was 10 minutes at 95° C., 50 cycles of 15 minutes at 95° C. and 1 minute at 60° C.

TABLE 5
Experiment on keratinocytes: relative expression of Sirt-1 mRNA

| | |
|---|---|
| Control (none) | 1.0 |
| Retinol | 7.9 |
| Green tea EGCG (10 ppm) | 8.2 |
| Green tea EGCG3″Me (10 ppm) | 17.1 |

TABLE 6
Experiment on fibroblasts: relative expression of Sirt-1 mRNA

| | |
|---|---|
| Control (none) | 1.0 |
| Retinol | 1.9 |
| Green tea EGCG (10 ppm) | 2.0 |
| Green tea EGCG3″Me (10 ppm) | 4.3 |

It was confirmed that the composition containing the methylated catechin as an active ingredient remarkably increases the expression of the Sirt-1 gene as compared to the unmethylated catechin.

(4) Evaluation of ERCC8 Activation

The effect of a methylated catechin on ERCC8 activation was compared with those of retinol and an unmethylated catechin.

Specifically, after treating the keratinocytes (NHK) and the fibroblasts (NHF) with each of retinol, green tea EGCG and green tea EGCG″3Me at 10 ppm, followed by incubation at 37° C. for 24 hours, total RNA was isolated from the cells and the relative expression of ERCC8 mRNA was compared.

The total RNA was isolated using TRIzol™ (Invitrogen, Carlsbad, Calif., USA) according to the manufacturer's protocol. RNA concentration was measured spectrophotometrically and RNA integrity was measured using BioAnalyzer 2100 (Agilent Technologies, Santa Clara, Calif., USA). 4 μg of RNA was reverse transcribed to cDNA using SuperScript®III reverse transcriptase (Invitrogen, Carlsbad, Calif., USA). The cDNA was stored at −70° C. The expression level of the target gene was measured by quantitative real-time TaqMan RT-PCR (7500Fast, Applied Biosystems, Foster City, Calif., USA). The cycle condition was 10 minutes at 95° C., 50 cycles of 15 minutes at 95° C. and 1 minute at 60° C.

TABLE 7
Experiment on keratinocytes: relative expression of ERCC8 mRNA

| | |
|---|---|
| Control (none) | 1.0 |
| Retinol | 1.2 |
| Green tea EGCG (10 ppm) | 1.9 |
| Green tea EGCG3″Me (10 ppm) | 4.3 |

TABLE 8
Experiment on fibroblasts: relative expression of ERCC8 mRNA

| | |
|---|---|
| Control (none) | 1.0 |
| Retinol | 1.0 |
| Green tea EGCG (10 ppm) | 2.3 |
| Green tea EGCG3″Me (10 ppm) | 5.1 |

It was confirmed that the composition containing the methylated catechin as an active ingredient remarkably increases the expression of the ERCC8 gene as compared to the unmethylated catechin.

(5) Evaluation of FoxO3 Activation

The effect of a methylated catechin on Fox03 activation was compared with those of retinol and an unmethylated catechin.

Specifically, after treating the keratinocytes (NHK) and the fibroblasts (NHF) with each of retinol, green tea EGCG and green tea EGCG″3Me at 10 ppm, followed by incubation at 37° C. for 24 hours, total RNA was isolated from the cells and the relative expression of Fox03 mRNA was compared.

The total RNA was isolated using TRIzol™ (Invitrogen, Carlsbad, Calif., USA) according to the manufacturer's protocol. RNA concentration was measured spectrophotometrically and RNA integrity was measured using BioAnalyzer 2100 (Agilent Technologies, Santa Clara, Calif., USA). 4 μg of RNA was reverse transcribed to cDNA using SuperScript®III reverse transcriptase (Invitrogen, Carlsbad, Calif., USA). The cDNA was stored at −70° C. The expression level of the target gene was measured by quantitative real-time TaqMan RT-PCR (7500Fast, Applied Biosystems, Foster City, Calif., USA). The cycle condition was 10 minutes at 95° C., 50 cycles of 15 minutes at 95° C. and 1 minute at 60° C.

TABLE 9
Experiment on keratinocytes: relative expression of Fox03 mRNA

| | |
|---|---|
| Control (none) | 1.0 |
| Retinol | 1.1 |
| Green tea EGCG (10 ppm) | 1.4 |
| Green tea EGCG3″Me (10 ppm) | 3.2 |

TABLE 10
Experiment on fibroblasts: relative expression of Fox03 mRNA

| | |
|---|---|
| Control (none) | 1.0 |
| Retinol | 1.4 |
| Green tea EGCG (10 ppm) | 2.1 |
| Green tea EGCG3″Me (10 ppm) | 5.3 |

It was confirmed that the composition containing the methylated catechin as an active ingredient remarkably increases the expression of the Fox03 gene as compared to the unmethylated catechin.

It confirmed that the composition according to the present disclosure, which contains the methylated catechin as an active ingredient, provides skin improving effect by moisturizing skin and strengthening skin barrier by activating the XPD gene, the Klotho gene, the Sirt-1 gene, the ERCC8 gene and the FoxO3 gene in keratinocytes which prevent evaporation of water and protect the skin from harmful factors. Also, it was confirmed that the composition provides antiaging effect by enhancing skin elasticity and improving skin wrinkles by activating the XPD gene, the Klotho gene, the Sirt-1 gene, the ERCC8 gene and the FoxO3 gene in fibroblast which are deeply associated with skin elasticity and skin wrinkles. These effects were remarkably superior as compared to the unmethylated catechin.

(6) Evaluation of Cellular Differentiation

As described below, it was confirmed that the methylated catechin which increases the expression of the XPD gene, the Klotho gene, the Sirt-1 gene, the ERCC8 gene and the FoxO3 gene can promote cellular differentiation by activating the longevity genes.

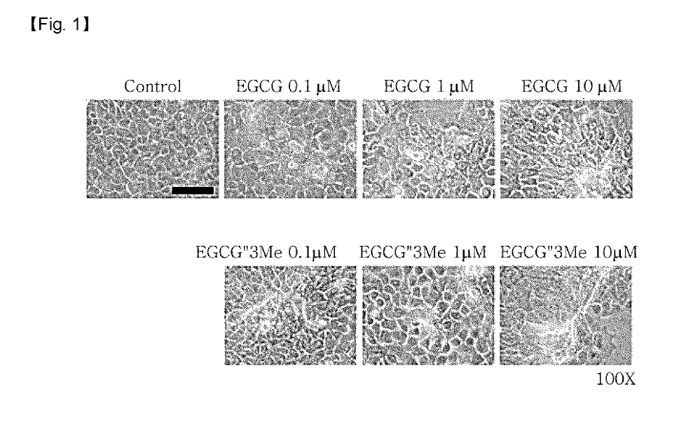

In order to investigate the effect of the methylated catechin on the differentiation of keratinocytes, normal human epidermal keratinocytes (NHEKs) were treated with EGCG″3Me and EGCG at various concentrations for 48 hours. As seen from FIG. 1 (scale bar=100 μm), EGCG″3Me promoted the differentiation of the keratinocytes in a concentration-dependent manner. It exhibited superior differentiation of the keratinocytes at low concentration as compared to EGCG. Accordingly, it can be seen that the methylated catechin is superior in terms of economy and skin improving effect such as skin moisturizing and skin barrier effects.

Also, it was found out that the methylated catechin has antiaging effect of enhancing skin elasticity and improving skin wrinkles by increasing extracellular matrices (ECM) in fibroblasts.

(7) Cell Survival Ratio

After treating normal human epidermal keratinocytes (NHEKs) with EGCG and EGCG″3Me at various concentrations (0, 0.1, 1, 10 and 50 μM), cell survival ratio was determined 48 hours and 72 hours later.

After treating the NHEKs with each of EGCG and EGCG″3Me for 48 hours and 72 hours, 50 μL (2 mg/mL) of thiazolyl blue tetrazolium bromide (MTT, Sigma-Aldrich, St. Louis, Mo., USA) dissolved in KGM-GOLD was added to the cells. After incubating at 37° C. for 3 hours, the medium was removed and the formazan crystals of the cells were softly shaken for 10 minutes and dissolved in 200 μL of DMSO. The quantity of the remaining formazan was measured at 540 nm using a microplate reader (Molecular Devices, Sunnyvale, Calif., USA).

It was found out that the methylated catechin which activates the expression of the longevity gene XPD shows higher cell survival ratio than the unmethylated catechin. In particular, the difference in cell survival ratio was significantly higher at low concentration. Accordingly, it can be seen that the methylated catechin is superior in terms of economy and efficiency.

Also, it was found out that the methylated catechin which activates the expression of the longevity gene Klotho shows higher cell survival ratio than the unmethylated catechin. In particular, the difference in cell survival ratio was significantly higher at low concentration. Accordingly, it can be seen that the methylated catechin is superior in terms of economy and efficiency.

Also, it was found out that the methylated catechin which activates the expression of the longevity gene Sirt-1 shows higher cell survival ratio than the unmethylated catechin. In particular, the difference in cell survival ratio was significantly higher at low concentration. Accordingly, it can be seen that the methylated catechin is superior in terms of economy and efficiency.

Also, it was found out that the methylated catechin which activates the expression of the longevity gene ERCC8 shows higher cell survival ratio than the unmethylated catechin. In particular, the difference in cell survival ratio was significantly higher at low concentration. Accordingly, it can be seen that the methylated catechin is superior in terms of economy and efficiency.

Also, it was found out that the methylated catechin which activates the expression of the longevity gene FoxO3 shows higher cell survival ratio than the unmethylated catechin. In particular, the difference in cell survival ratio was significantly higher at low concentration. Accordingly, it can be seen that the methylated catechin is superior in terms of economy and efficiency.

(8) Evaluation of Safety for Skin

The safety for skin of the composition according to the present disclosure was evaluated by measuring skin irritation of a cosmetic composition of Formulation Example 9.

It was found out that the cosmetic composition of Formulation Example 9 according to the present disclosure shows superior safety for skin without causing skin irritation in any of 30 adult subjects who applied it.

Hereinafter, formulation examples of the composition according to the present disclosure are described. However, other types of formulations are also possible and the scope of the present disclosure is not limited by them.

### [Formulation Example 1] Health Food

| | | |
|---|---|---|
| Green tea EGCG3″Me | 1000 | mg |
| Vitamin mixture | | |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Nicotinamide | 1.7 | mg |
| Folic acid | 50 | μg |
| Calcium pantothenate | 0.5 | mg |
| Mineral mixture | | |
| Ferrous sulfate | 1.75 | mg |
| Zinc oxide | 0.82 | mg |
| Magnesium carbonate | 25.3 | mg |
| Potassium phosphate monobasic | 15 | mg |
| Calcium phosphate dibasic | 55 | mg |
| Potassium citrate | 90 | mg |
| Calcium carbonate | 100 | mg |
| Magnesium chloride | 24.8 | mg |

The compositional ratios of the vitamin and mineral mixtures described above are given as specific examples relatively appropriate for a health food but may be varied as desired.

### [Formulation Example 2] Health Drink

| | |
|---|---|
| Green tea EGCG3″Me | 1000 mg |
| Citric acid | 1000 mg |
| Oligosaccharide | 100 g |
| Taurine | 1 g |

According to a common health drink preparation method, the above-described ingredients were mixed and purified water was added to make a final volume 900 mL. After heating at 85° C. for about 1 hour under stirring, the resulting solution was filtered and sterilized. The compositional ratio is given as a specific example relatively appropriate for a health drink but may be varied as desired taking into account regional and ethnic preferences such as particular consumers, countries, purpose of use, etc.

### [Formulation Example 3] Powder

A powder was prepared by mixing 20 mg of green tea EGCG3″Me powder, 100 mg of lactose and 10 mg of talc and filling in a pouch.

### [Formulation Example 4] Tablet

10 mg of green tea EGCG3″Me powder, 100 mg of cornstarch, 100 mg of lactose and 2 mg of magnesium stearate were mixed. The mixture was prepared into a tablet according to a common tablet making method.

### [Formulation Example 5] Capsule

A capsule was prepared according to a common capsule preparation method by mixing 10 mg of green tea EGCG3″Me powder, 3 mg of crystalline cellulose, 14.8 mg of lactose and 0.2 mg of magnesium stearate and filling in a gelatin capsule.

### [Formulation Example 6] Injection

An injection was prepared according to a common injection preparation method by mixing 10 mg of green tea EGCG3″Me powder, 180 mg of mannitol, 2974 mg of sterile distilled water for injection and 26 mg of Na₂HPO₄.12H₂O per ampoule (2 mL).

### [Formulation Example 7] Liquid

According to a common liquid preparation method, 20 mg of green tea EGCG3″Me powder, 10 g of high-fructose corn syrup, 5 g of mannitol and an adequate amount of purified water were dissolved by adding to purified water. After making a final volume 100 mL by adding purified water, the liquid was filled in a brown bottle and then sterilized.

### [Formulation Example 8] Ointment

An ointment was prepared according to a common method with the following composition (unit: wt %).

| | |
|---|---|
| Green tea EGCG3″Me | 3.0 |
| Glycerin | 8.0 |
| Butylene glycol | 4.0 |
| Liquid paraffin | 15.0 |
| β-Glucan | 7.0 |
| Carbomer | 0.1 |
| Caprylic/capric triglyceride | 3.0 |
| Squalane | 1.0 |
| Cetearyl glucoside | 1.5 |
| Srbitan stearate | 0.4 |
| Cetearyl alcohol | 1.0 |
| Beswax | 4.0 |
| Antiseptic, colorant and fragrance | adequate |
| Purified water | balance |

### [Formulation Example 9] Nourishing Lotion (Milk Lotion)

A nourishing lotion was prepared according to a common method with the composition described in Table 11.

TABLE 11

| Ingredients | Contents (wt %) |
|---|---|
| Green tea EGCG3″Me | 0.1 |
| Glycerin | 3.0 |
| Butylene glycol | 3.0 |
| Propylene glycol | 3.0 |
| Carboxyvinyl polymer | 0.1 |
| Beeswax | 4.0 |
| Polysorbate 60 | 1.5 |
| Caprylic/capric triglyceride | 5.0 |
| Squalane | 5.0 |
| Sorbitan sesquioleate | 1.5 |
| Cetearyl alcohol | 1.0 |
| Tromethamine | 0.2 |
| Antiseptic and fragrance | adequate |
| Purified water | balance |
| Total | 100 |

### [Formulation Example 10] Nourishing Cream

A nourishing cream was prepared according to a common method with the composition described in Table 12.

TABLE 12

| Ingredients | Contents (wt %) |
|---|---|
| Green tea EGCG3″Me | 0.1 |
| Glycerin | 3.5 |
| Butylene glycol | 3.0 |
| Liquid paraffin | 7.0 |
| β-Glucan | 7.0 |
| Carbomer | 0.1 |
| Caprylic/capric triglyceride | 3.0 |
| Squalane | 5.0 |
| Cetearyl glucoside | 1.5 |
| Sorbitan stearate | 0.4 |
| Polysorbate 60 | 1.2 |
| Tromethamine | 0.1 |
| Antiseptic and fragrance | adequate |
| Purified water | balance |
| Total | 100 |

While the exemplary embodiments have been shown and described, it will be understood by those skilled in the art that various changes in form and details may be made thereto without departing from the spirit and scope of this disclosure as defined by the appended claims. In addition, many modifications can be made to adapt a particular situation or material to the teachings of this disclosure without departing from the essential scope thereof. Therefore, it is intended that this disclosure not be limited to the particular exemplary embodiments disclosed as the best mode contemplated for carrying out this disclosure, but that this disclosure will include all embodiments falling within the scope of the appended claims.

1. A method for activating longevity genes of a subject, wherein the method comprises administering an effective amount of a methylated catechin, a salt thereof, a prodrug thereof, a hydrate thereof, a solvate thereof or an isomer thereof to a subject in need thereof, wherein the longevity gene is one or more of an XPD gene, a Klotho gene, a Sirt-1 gene, an ERCC8 gene and a FoxO3 gene.

2. The method according to claim 1, wherein the activation of the longevity gene enhances transcription to mRNA.

3. The method according to claim 1, wherein the methylated catechin is extracted from green tea leaf.

4. The method according to claim 1, wherein the methylated catechin is represented by Chemical Formula 1: 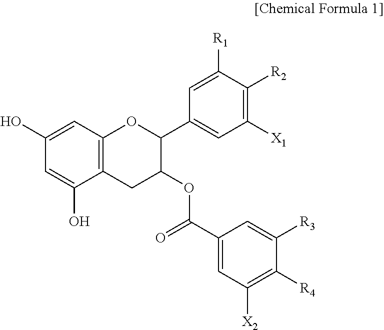
wherein
each of R1, R2, R3 and R4 is independently OCH3 or OH, except for the case where all of R1, R2, R3 and R4 are OH, and
each of X1 and X2 is independently H or OH.

5. The method according to claim 1, wherein the methylated catechin is one or more selected from a group consisting of EGCG3″Me (epigallocatechin-3-O-(3-O-methyl)gallate), EGCG4″Me (epigallocatechin-3-O-(4-O-methyl)gallate), ECG3″Me (epicatechin-3-O-(3-O-methyl)gallate), ECG4″Me (epicatechin-3-0-(4-O-methyl)gallate), GCG3″Me (gallocatechin-3-O-(3-O-methyl)gallate), GCG4″Me (gallocatechin-3-O-(4-O-methyl)gallate), CG3″Me (catechin-3-O-(3-O-methyl)gallate) and CG4″Me (catechin-3-O-(4-O-methyl)gallate).

6. The method according to claim 5, wherein the methylated catechin is EGCG3″Me (epigallocatechin-3-O-(3-O-methyl)gallate).

7. The method according to claim 1, wherein the methylated catechin or the salt, prodrug, hydrate, solvate or isomer thereof is administered in a form of a composition, wherein the composition comprises 0.0001-10 wt % of the methylated catechin, the salt thereof, the prodrug thereof, the hydrate thereof, the solvate thereof or the isomer thereof, based on the total weight of the composition.

8. The method according to claim 1, wherein the method is for enhancing the expression of one or more of an XPD protein, a Klotho protein, a Sirt-1 protein, an ERCC8 protein and a FoxO3 protein.

9. The method according to claim 1, wherein the method is for extending life span delaying biological or skin aging, or improving symptoms of biological or skin aging.

10. The method according to claim 9, wherein the method is for enhancing skin elasticity or improving skin wrinkles.

11. The method according to claim 1, wherein the method is for improving skin.

12. The method according to claim 11, wherein the method is for moisturizing skin or strengthening skin barrier.

13. The method according to claim 1, wherein the method is for preventing or treating a one or more of an XPD-related disease, a Klotho-related disease, a Sirt-1-related disease, an ERCC8-related disease and a FoxO3-related disease.

14. The method according to claim 13, wherein the XPD-related disease is cancer, xeroderma pigmentosum, Cockayne syndrome or trichothiodystrophy, the Klotho-related disease is arteriosclerosis, osteoporosis, stroke or Alzheimer's disease, the Sirt-1-related disease is cancer, diabetes, neurodegenerative disease, obesity, inflammatory disease or allergic respiratory disease, the ERCC8-related disease is cancer or Cockayne syndrome, and the FoxO3-related disease is cancer or inflammatory disease.

15. The method according to claim 1, wherein the methylated catechin or the salt, prodrug, hydrate, solvate or isomer thereof is administered in a form of a pharmaceutical composition.

16. The method according to claim 1, wherein the methylated catechin or the salt, prodrug, hydrate, solvate or isomer thereof is administered in a form of a cosmetic composition.

17. The method according to claim 1, wherein the methylated catechin or the salt, prodrug, hydrate, solvate or isomer thereof is administered in a form of a food composition.
